(19) United States
(12) Patent Application Publication
(10) Pub. No.: US 2017/0132393 A1
(43) Pub. Date: May 11, 2017

(54) PRESCRIPTION HOME DELIVERY

(71) Applicant: Wal-Mart Stores, Inc., Bentonville, AR (US)

(72) Inventors: Chandrashekar Natarajan, San Ramon, CA (US); Donald R. High, Noel, MO (US); John J. O'Brien, V, Farmington, AR (US)

(21) Appl. No.: 15/348,449

(22) Filed: Nov. 10, 2016

Related U.S. Application Data
(60) Provisional application No. 62/253,534, filed on Nov. 10, 2015.

(51) Int. Cl. G06F 19/00; G06Q 10/08

(52) U.S. Cl. CPC G06F 19/3456; G06Q 10/087

(57) ABSTRACT

A method and system for home delivery of prescribed medication comprise processing a prescription for a medication at a first location, purchasing at least one item of interest at a second location, and combining the prescribed medication and the at least one item of interest under a same delivery to a third location determined by a user of the prescribed medication and at least one item of interest.

### RELATED APPLICATIONS

This application claims priority to U.S. Provisional Patent Application No. 62/253,534, filed Nov. 10, 2015 and entitled “Prescription Home Delivery,” the entirety of which is incorporated by reference herein.

### FIELD

The present concepts relate generally to the delivery of pharmacy items, and more specifically, to a prescription delivery service.

### BACKGROUND

It is well-known that people typically visit a doctor's office when they are ill. In cases where medicine is prescribed, the patient may visit a pharmacy for filling the prescription. The patient may also visit a grocery store or other retail establishment for purchasing food or other consumer goods of interest, so that the patient prescribed the medication is prepared for a home stay and does not need to make subsequent trips to the store while sick.

### BRIEF SUMMARY

In one aspect, provided is a method for home delivery of prescribed medication, comprising processing a prescription for a medication at a first location; purchasing at least one item of interest at a second location; and combining the prescribed medication and the at least one item of interest under a same delivery to a third location determined by a user of the prescribed medication and at least one item of interest.

In some embodiments, the first location is a pharmacy and the second location is a retail establishment. In some embodiments, the pharmacy and the retail establishment are at a same location, or are the same.

In some embodiments, the method further comprises sending the prescription via electronic communications from a doctor's office to the first location; and generating a recommendation for other items of interest based on the prescription.

In some embodiments, the method further comprises generating an automatic replenishment of the prescription based on the prescription.

In some embodiments, the method further comprises generating an alert that is sent to the first location when an inventory supply falls below a predetermined threshold.

In some embodiments, the method further comprises identifying a plurality of pharmacies available for processing the prescription; and comparing a current location of the user and the first location to identify a pharmacy of the plurality of pharmacies for processing the prescription.

In some embodiments, the method further comprises delivering the prescribed medication and the at least one item of interest in a temperature controlled device. In some embodiments, a delivery encryption system comprises a blockchain for package tracking and authentication.

In some embodiments, the method further comprises delivering the prescribed medication and the at least one item of interest in an autonomous vehicle.

In another aspect, provided is a prescription processing system, comprising: an inventory determination processor that monitors an availability of a medication for prescribing to a recipient; and a related products generator that determines items that may be combined with a prescription of the medication and other items that can be delivered together under a same shipping order to the recipient.

In some embodiments, the prescription processing system further comprises a pharmacy determination processor 34 can identify a plurality of pharmacies may be available for prescribing a medication.

In some embodiments, the prescription processing system further comprises a product recommendation processor that compiles data related to buying habits of the recipient and compares this data to the medication prescription to generate recommended buying options.

In some embodiments, the prescription processing system further comprises a medication refill tracking processor that monitors refill dates regarding the prescription.

In some embodiments, the prescription processing system determines a delivery by combining the prescribed medication at a first location and the other items at a second location, and wherein the delivery is made to a third location.

In some embodiments, the prescription processing system further includes a blockchain processor for exchanging delivery information with one or more supply chain entities, wherein the blockchain processor is part of or in communication with a ledger system for communicating with one or more electronic devices of a customer, doctor, pharmacy, courier, and supply chain entities.

In another aspect, provided is a method for home delivery of prescribed medication, comprising: processing a prescription for a medication; determining whether to purchase at least one item of interest; combining the prescribed medication and the at least one item of interest under a same delivery to a predetermined location in response to determining that the at least one item of interest is purchased; and delivering on the prescribed medication to the predetermined location in response to determining that the at least one item of interest is not purchased.

In some embodiments, the medication is at a first location and the at least one item of interest is at a second location. In some embodiments, the first location is a pharmacy and the second location is a retail establishment.

In some embodiments, the medication and the at least one item of interest are each at a same location.

In some embodiments, the method further comprises generating an automatic replenishment of the prescription based on the prescription.

In some embodiments, the method further comprises generating an alert when an inventory supply falls below a predetermined threshold.

In some embodiments, the method further comprises identifying a plurality of pharmacies available for processing the prescription; and comparing a current location of a user and a location of the medication to identify a pharmacy of the plurality of pharmacies for processing the prescription.

### BRIEF DESCRIPTION OF THE SEVERAL VIEWS OF THE DRAWINGS

The above and further advantages may be better understood by referring to the following description in conjunction with the accompanying drawings, in which like numerals indicate like structural elements and features in various figures. The drawings are not necessarily to scale, emphasis instead being placed upon illustrating the principles of the concepts.

FIG. 1 is a diagram of an environment in which embodiments may be practiced.

FIG. 2 is a block diagram of a prescription processing system, in accordance with some embodiments.

FIG. 3 is a flowchart of a method for delivering a prescribed medication to a consumer's home, in accordance with some embodiments.

FIG. 4 is a process flow for a prescription delivery service, in accordance with some embodiments.

FIG. 5 is a process flow for processing patient records and prescription data, in accordance with some embodiments.

FIG. 6 is a process flow for pharmacy fulfillment of a prescription, in accordance with some embodiments.

FIG. 7 is a patient decision process flow after a prescription fulfillment, in accordance with some embodiments.

FIG. 8 is a process flow for a prescription fulfillment, in accordance with some embodiments.

FIG. 9 is a process flow for a prescription fulfillment, in accordance with some embodiments.

### DETAILED DESCRIPTION

Systems and methods in accordance with some embodiments provide for a combined delivery of medication prescriptions and groceries or other items of interest to a desired location.

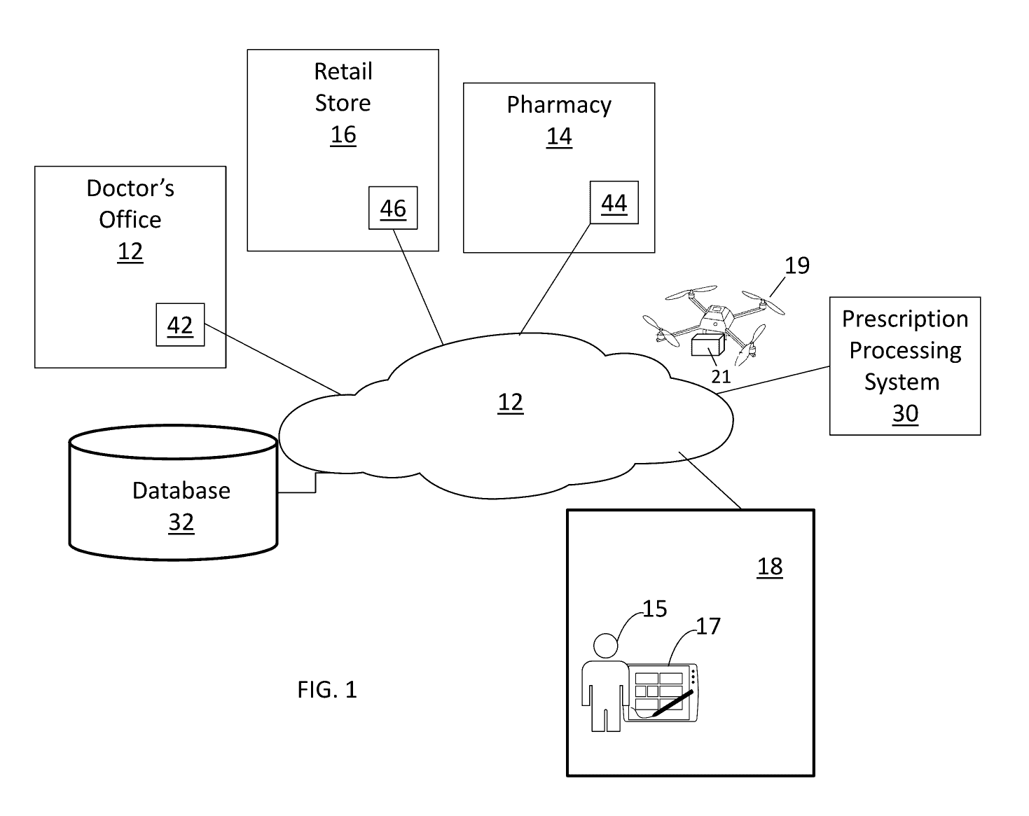

FIG. 1 is a diagram of an environment in which embodiments may be practiced. The environment may include entities involved in the delivery of pharmacy items and groceries or other retail items, in accordance with some embodiments. Entities may include but not be limited to a doctor's office 12, pharmacy 14, grocery store 16, and user location 18 such as a home or office. The environment may include an autonomous vehicle 19 such as a driverless, self-driving, or robotic vehicle, unmanned aerial vehicle (UAV), or the like, which can deliver the pharmacy items and groceries or other retail items between the various entities. In other embodiments, pharmacy items and groceries or other retail items are delivered by conventional vehicles, e.g., automobiles, trucks, and so on. In some embodiments, the information required for delivering a prescription may be transmitted and authenticated through a peer-peer ledger system, which will allow the autonomous vehicle to receive pickup information and where to deliver the prescription to; as well as authentication for the vehicle and authentication of the customer when receiving the package.

Each of the doctor's office 12, pharmacy 14, grocery store 16, and user location 18 may include at least one computer processor 17, 42, 44, 46 and network interface for communicating with each other and/or other electronic devices such as a prescription processing system 30 and database 32 via a network 12. The network 12 may be a public switched telephone network (PSTN), a mobile communications network, a data network, such as a local area network (LAN) or wide area network (WAN), or a combination thereof, or other communication network known to those of ordinary skill in the art.

A consumer 15 may visit the doctor's office 12, or a hospital, medical center, or other location where the consumer 15 may receive a prescription. For purposes of explanation, a consumer 15 may also be referred to as a medication recipient or recipient when the consumer 15 is intended to be the recipient of prescribed medication. The consumer 15 may then visit the pharmacy 14 to receive the medication identified in the prescription. The consumer 15 may subsequently visit a retail establishment 16 such as a grocery store to purchase other items of interest, which may or may not relate to the medication acquired at the pharmacy 14. In some embodiments, these items may be purchased that the pharmacy 14 instead of a physically different store. In some embodiments, the consumer 15 may fill a prescription online, and also purchase the other items online, for example, at a same or different websites.

The prescription processing system 30 provides for the delivery of a medication prescription to a desired receiving location 18 of a recipient 15 such as a home, and identifies other items such as groceries, supplies, or other retail items that can be bundled and delivered with the medication prescription to the desired receiving location 18. In the foregoing example, a consumer 15 visiting the pharmacy 14 and/or store 16 may desire to ship the medication and other items under a same shipment, for example, in instances where the medication is not available at the time of the request made by the consumer 15 at the pharmacy 14. In other embodiments, the consumer 15 may order the medication online using a smartphone or other computer, for example, refills, along with other items, whereby the medication and items can be bundled and shipped together to a location of preference.

The prescription processing system 30 may offer additional features such as automatic replenishment of a prescription and/or purchase recommendations based on prescription information, generating alerts sent to the pharmacy 14 when inventory supplies fall below a predetermined threshold, cross-selling, monitoring, advertising, notifications, and other related features. Alerts can be sent from the peer-peer ledger system, which is communicated to the person's smart device.

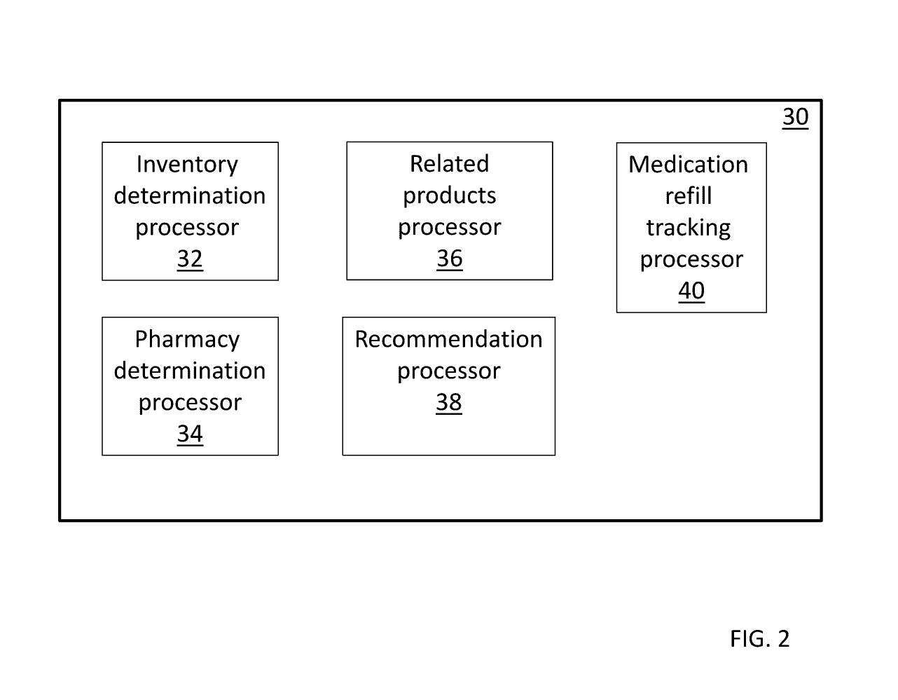

FIG. 2 is a block diagram of a prescription processing system 30, in accordance with some embodiments. The prescription processing system 30 can be implemented in the environment illustrated and described with respect to FIG. 1.

The prescription processing system 30 may include an inventory determination processor 32, a pharmacy determination processor 34, a related products generator 36, a product recommendation processor 38, and a medication refill tracking processor 40. Some or all of these elements of the prescription processing system 30 may be present under a same computer hardware platform. In other embodiments, these elements may be located on two or more different computer hardware platform, and can communicate with each other and/or other elements of the prescription processing system 30 via the communication network 12, for example, wired or wireless network that exchanges data electronically.

The inventory determination processor 32 keeps track of medication availability. In doing so, the inventory determination processor 32 may receive a communication from a pharmacy processor 44 when a consumer's medication prescription is processed, whereby a predetermined amount of medication is allotted to the consumer recipient 15. For example, a consumer 15 may receive a prescription from a doctor. In doing so, the order is entered into the doctor office processor 42 and submitted to the pharmacy 14. The inventory determination processor 32 may receive this information from the doctor's processor 42 and/or the pharmacy processor 44. The inventory determination processor 32 can establish from this information an amount of available medication at the pharmacy 14. The inventory determination processor 32 can check for the availability of inventory. Here, the consumer 15 can access the program, for example, by opt into the program. The doctor office processor 42 may process the prescription information, then send the prescription information to the pharmacy processor 44 along with customer information.

The prescription processing system 30 may include one or more processors for performing blockchain-related processing, and may further include a communication device that is linked to a peer-to-peer network, with affiliated keys for patient, doctor, and pharmacy distributed to a peer-to-peer network, which further communicates this information to the various entities. At each transmission of communication a new block may be formed with the subsequent blocks; which also includes handling, procurement, issuing, or acceptance of the prescription. Prescriptions and their related ingredients, products and their related inventory, retailer and their related inventory, and pharmacy and their related inventory, will share their inventory statuses with the application or the blockchain structure, as blocks of information to the peer-to-peer ledger system, or as inventory statuses with the application.

The doctor office processor 42 and/or other business entity computer device affiliated with the doctor may provide an authentication or other security technique including public and/or private keys when submitted prescriptions in the form of electronic data to other entities, such as the pharmacy 14. Here, the consumer 15, e.g., a patient of the doctor, may also having a public and/or private key for communicating with the consumer's computer device and the doctor office processor 42 and/or other business entity computer device. When the prescription is sent from the doctor office processor 42 or their affiliation to the pharmacy 14 it may include a converged blockchain structure of both the doctor's private/public key and the patient's private/public key. This information will be shared on a peer-to-peer network, where the pharmacy 14 has access to the data, provided the pharmacy's key has been granted access to the prescription.

After the pharmacy processor 44 has authenticated and decrypted the prescription chain, a receiver for example at the pharmacy 14 may view and produce the prescription, which will further alter the blockchain to include but not be limited to the information from the pharmacy, pharmacist, date, time, prescription instructions, dosage instructions, special instructions, handlings, pickup information, authentication, class of drug, or a combination thereof.

Products and services available at the retailer may also have unique blockchain identifiers, which may be aligned with or otherwise related to prescriptions for relevant combination purchasing, which may be contained and referenced from a database.

Through the application or through a peer-to-peer ledger system, referral products will be submitted to the customer, where the customer can opt-in or opt-out of referral purchases.

The system 30 may also alert the consumer 15, e.g., a patient of the doctor, of which pharmacy the consumer 15 will receive the order. The inventory determination processor 32 in coordination with the refill tracking processor 40 can monitor prescription information, such as refill dates and so on. Refill dates are a part of the pharmacy information sent to the pharmacy 14. The purpose of refill dates is to enable to pharmacy 14 to better predict the need of pharmaceuticals. In some embodiments, refills are accomplished through blockchain data that contains information on refill dates, etc. Alerts may be parsed when refill dates have been achieved from the ledger system to the person's chosen device or method.

The pharmacy determination processor 34 can identify a plurality of pharmacies may be available for prescribing a medication. The pharmacy determination processor 34 can compare a current location of the recipient 15, for example, by GPS communicating with the recipient's smartphone 17, to the location of different pharmacies, and provide the result to the pharmacy determination processor 34, which can recommend the closest pharmacy, or other parameter, for example, the largest pharmacy, the pharmacy where the consumer 15 has previous relationship, the pharmacy having other goods of interest such as food that can be bundled with the medication. Pharmacy providers may have their information stored on a peer-peer ledger system. Thus, when a prescription has been sent by a doctor to the patient's record, the patient will have options to view and select pharmacies. Once selected, the prescription will be transmitted to the chosen pharmacy.

The identification of a pharmacy location based on proximity can be determined through the patient and/or pharmacy blockchain profile, which will include information on the geolocation relating to their position. Further, information relating to the expected time of completion of a prescription may also be included. Each of the above may be produced and distributed through the application and its databases, or through the blockchain and a peer-to-peer ledger system. In doing so, the prescription processing system 30 may include a blockchain processor for exchanging delivery information with one or more supply chain entities, wherein the blockchain processor is part of or in communication with a ledger system for communicating with one or more electronic devices of a customer, doctor, pharmacy, courier, and supply chain entities. In other embodiments, blockchain processing is performed external to the prescription processing system 30 or in combination with the blockchain processor of the prescription processing system 30.

The related products processor 36 determines items that may be combined with prescribed medicine so that the prescribed medication and items can be shopped together under a same shipping order to the consumer 15. An association may be established. For example, a prescription has directions, which both the doctor's office computer 42 and the pharmacy computer 44 may store and process. Directions may include a request for the recipient to take with water, milk, juice, or without certain products. The system 30 uses this information to create recommended products to the consumer 15. The consumer 15 can customize the groceries or other items that the consumer 15 desires to receive along with the prescription. The related products processor 36 may receive a shopping list from the shopper 15, i.e., provided electronically from a smartphone 17 or other electronic device.

Couriers, delivery agents, and delivery providers, or the like may distribute information relating to their location, service, availability, and estimated time of completion, with the application. Couriers, delivery agents, and delivery providers, or the like may distribute information relating to their location, service, availability, and estimated time of completion, with the blockchain peer-peer system. To perform a blockchain method in accordance with some embodiments, this will require blockchain private and public keys issues to couriers, delivery agents, delivery providers, or the like.

The recommendation processor 38 compiles data related to buying habits of the consumer 15 and compares this data to the medication prescription to generate recommended buying options. In addition, the recommendation processor 38 can notify the recipient 15 of products being bought that will complement the medication, or conflict with the medication, so that the recipient 15 may select, for example, from a computer user interface, items of interest which may be bundled with the prescribed medication. For example, the recommendation processor 38 can recommend products that may benefit the health of the consumer 15, for example, low sodium foods that may be consumed for the consumer taking cholesterol medication, or sugar free alternative items for consumers taking diabetes medication.

The recommendation processor 38 may also send an alert to the pharmacy processor 44 when a determination is made by the inventory determination processor 32. In some embodiments, refill levels are estimated by the inventory determination processor 32 based upon the purchase date, prescribed dosage per day, quantity in the bottle, or other related information. For example, a 30 day supply starting on the beginning of the month with 1 a day dosage and having 30 pills would indicate the customer would run out by the end of the month.

In some embodiments, alerts are determined from the block of information relating to a prescription, which is provided by the doctor or their affiliates, or the pharmacy. In some embodiments, alerts include time-based, having incremental windows approaching the expiration or replenishment of a prescription. Alerts may be threshold-based on the level of prescription inventory found in a connected device, such as IoT; where further thresholds determine when a product should warrant an alert to the customer. Alerts may provide autonomous reordering or fulfillment of prescription drugs. Alerts may be issued after a product has depleted. Alerts may be shared through the application of this system or distributed through the peer-to-peer ledger system used with a blockchain.

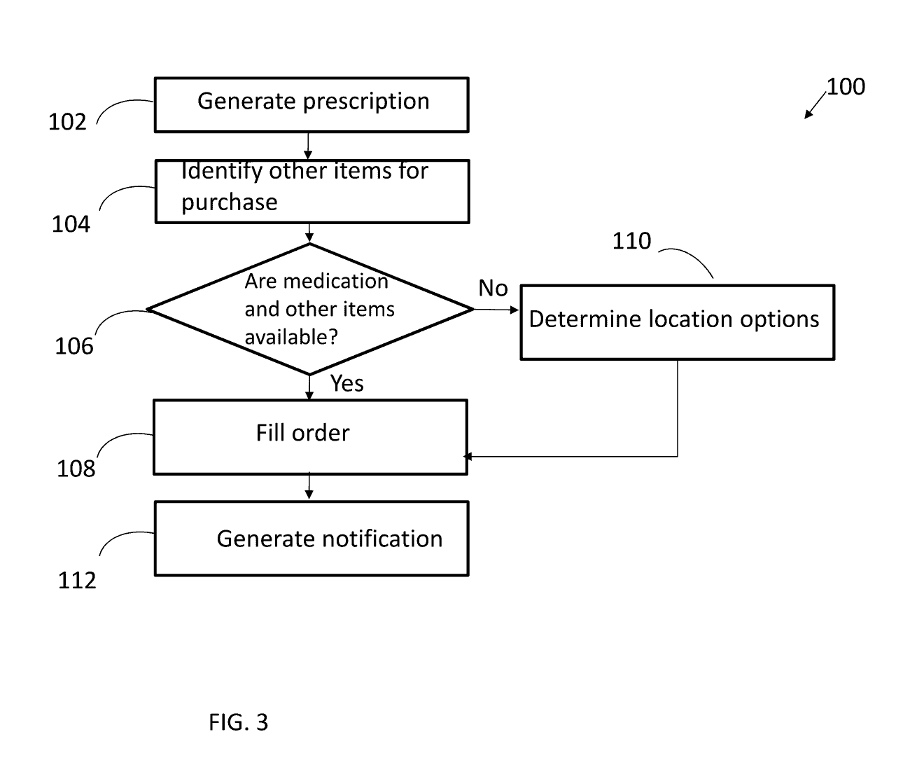

FIG. 3 is a flowchart of a method 100 for delivering a prescribed medication to a consumer's home, in accordance with some embodiments. Some or all of the method 100 can be performed at the prescription processing system 30 of FIGS. 1 and 2, and/or other elements of the environment illustrated in FIG. 1. The method 100 can be governed by instructions that are stored in a memory device of the prescription processing system 30 of FIGS. 1 and 2, and/or other elements of the environment illustrated in FIG. 1, and executed by a hardware processor of the prescription processing system 30 of FIGS. 1 and 2, and/or other elements of the environment illustrated in FIG. 1.

At block 102, a prescription is generated, for example, by a doctor, pharmacist, or other authorized party. The prescription can be filled at a pharmacy, hospital, medical center, or the like. The prescription can be presented at the pharmacy 14 by the medication recipient 15 who receives the prescription at the doctor's office 12, or the prescription can be electronically delivered from the doctor's office computer 42 to the pharmacy computer 44.

At block 104, the prescription recipient 15 identifies additional items for purchase contemporaneously with the filling of the prescription. The prescription recipient 15 may desire to purchase groceries or other products that are related to the prescribed medication, or otherwise purchased for other reasons, for example, convenience on the part of the recipient 15. The sequential order may vary. For example, the items may be purchased before the prescription is filled, or vice versa as shown.

At decision diamond 106, a determination is made whether there is sufficient inventory at the pharmacy 14 at which the request for prescription filling is made. Similarly a determination is made whether the item of interest is available at the store 16 visited by the consumer 15. In some embodiments where the purchase is made online, for example, an e-commerce purchase, a determination is made whether the order can be fulfilled online, for example, by the website at which the order is made. Orders made online may be fulfilled at multiple locations due to one store not having the product and the order being transferred and consolidated at one location to further the capability to deliver products efficiently.

If either the prescription or the other items of interest are not available at their respective locations of initial purchase, at block 110, the consumer 15 may consider different options. One option is for the consumer 15 to purchase the medication and other items of interest from a different store than the pharmacy 14 or the store 16. Another option is to acquire the medication and other items from different locations. As described herein, the prescription processing system 30 can reconcile the delivery of the medication and other items, regardless of whether they are purchased from different stores. Another option is to only purchase the medication from the pharmacy 14 and to forgo the purchase of other items.

At block 108, the order is filled. This includes the coordinated delivery of the medication and items of interest under a single delivery order.

At block 112, a notification is generated by the prescription processing system 30 indicating of the delivery of the prescribed medication and other items. Other notifications may include recommendations on additional items that may be of interest to the consumer 15, for example, a cross-selling feature, or warnings related to possible conflicts between the medication and the other items.

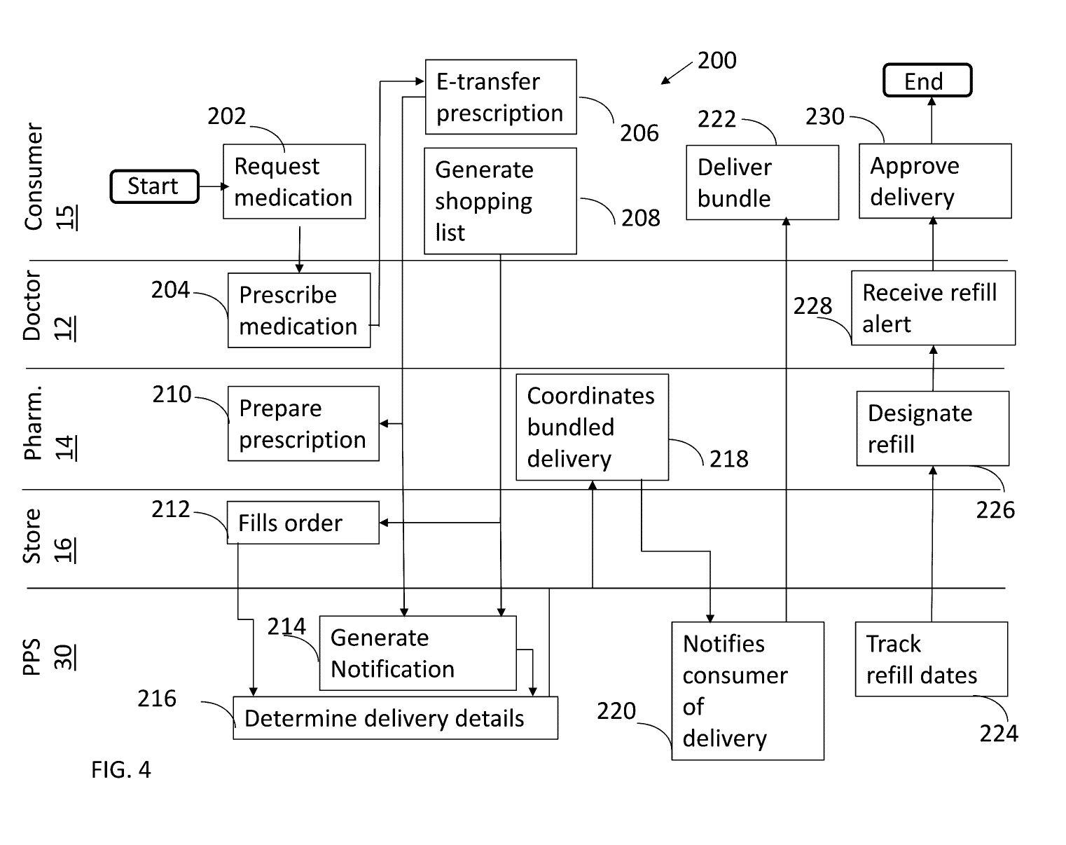

FIG. 4 is a process flow 200 for a prescription delivery service, in accordance with some embodiments. Some or all of the method 100 can be performed at the prescription processing system 30 of FIGS. 1 and 2, and/or other elements of the environment illustrated in FIG. 1. The method 100 can be governed by instructions that are stored in a memory device of the prescription processing system 30 of FIGS. 1 and 2, and/or other elements of the environment illustrated in FIG. 1, and executed by a hardware processor of the prescription processing system 30 of FIGS. 1 and 2, and/or other elements of the environment illustrated in FIG. 1.

At block 202, a source of medication is requested for a customer 15. The request may be made by the customer 15, or another authority may request medication on behalf of the customer 15, for example, a doctor. Accordingly, at block 204, a doctor 12 prepares a prescription for the requested medication.

The consumer 15 may receive an electronic version of the prescription, for example, generated by the doctor's computer 42. At block 206, the prescription is provided to the pharmacy 14 for preparing (block 210) the prescription. In some embodiments, the prescription is automatically transferred to the pharmacy 14.

At block 208, the consumer 15 generates a shopping list, for example, by executing a software application and entering the list electronically on an electronic device 17.

At block 212, a store 16 fills an order corresponding to the items on the shopping list.

At block 214, a notification may be generated in response to the prescription processing system 30 receiving data related to at least one of the prescription or the shopping list. The notification can include recommendations on additional items that may be of interest to the consumer 15 based on the prescribed medication. For example, a recommendation to purchase bottled water may be generated when the prescription includes medication in a pill format. Other notification may relate to a conflict between the purchased items on the shopping list and the prescribed medication. For example, a warning may be generated when the shopping list includes alcohol, and the prescribed medication includes instructions not to consume alcohol, noting that the prescription processing system 30 may store the medication instructions or retrieve medication instructions from the pharmacy computer 44 or other remote storage device.

At block 216, the prescription processing system 30 may determine delivery details with respect to the purchased medication and additional items, regardless of whether the medication and additional items are purchased together, for example, at the pharmacy, or purchased separately at different stores. The prescription processing system 30 may provide a mailing address and other information to the pharmacy 14, store 16, and/or other entity shipping the medication and the other items. At block 218, the pharmacy computer 44 receives the bundled shipment information from the prescription processing system 30 and coordinates with the store 16, for example, via the prescription processing system 30, to ship the prescription and other items together under a single order in accordance with the bundled shipment information, so that the consumer 15 pays for a single delivery. In other embodiments, the pharmacy 14 and store 16 communicate with each other by phone, email, or other communication.

At block 220, the prescription processing system 30 generates a notification that can sent to an electronic device 17 of the consumer 15 indicating of the delivery of the prescribed medication and other items.

At block 222, the medication and other items are delivered together at the predetermined delivery location. The medication and other items can be placed in a special package that addresses requirements of the medication and/or other items, for, a temperature controlled box 21 (see FIG. 1) or related container for housing the medication during delivery. Although a box 21 is referred to, other storage devices may equally apply, for example, containers that are configured differently than a square or rectangular box. Although the medication and items are delivered together, they may be placed in the same or separate packaging, for example, different containers.

The special package, or temperature controlled box 21, for example, may include an inner volume for storing the medication and/or other items during delivery by the vehicle 19, and a door for opening and closing the box 21, i.e., exposing the inner volume or enclosing the items therein. The box 21 may include one or more adapters, sensors, monitors, regulators, controllers, and so on. The box 21 may be part of or otherwise configured to deliver items of interest between locations in the autonomous vehicle 19.

For example, an adapter may be coupled to an air conditioning unit and/or heating unit for cooling and/or heating the inner volume of the box or otherwise providing a desired temperature Other adapters may be coupled to a temperature regulator, humidity regulator, a power source, security panel, and so on for providing a desired environmental feature.

In some embodiments, the box 21 may include an internet of things (IoT) device or computer interface that allows a user to log in and set the temperature, or to configure the system so that the temperature is adjusted automatically, for example, a temperature controller that changes the temperature to accommodate the package placed in a compartment or sub-compartment of the inner volume of the box 21 in response to temperature readings provided by a temperature, pressure, humidity, and/or other sensor. The temperature controller may generate temperature information on a regular basis to an electronic device in possession by a user, for example, output temperature information regarding the interior of a storage compartment every 5 minutes to the prescription processing system 30 and/or computer servers 42, 44, and 46.

As described herein, the inner volume, or storage compartment, of the box 21 may be temperature-controlled, for example, heated or cooled. In some embodiments where the storage compartment includes multiple sections or sub-compartments, the sub-compartments are maintained at one temperature by a sensor, while another sub-compartment is maintained at a different temperature.

In another example, the box 21 may include a monitoring device that controls and monitors an operation of the box 21, for example, environmental conditions and status of contents, a power source, location tracking, access and security, and so on. Thus the box 21 may monitor medication and/or store items that may include perishable goods, such as milk. In some embodiments, the box 21 includes a communication device for communicating with the vehicle 19. For example, a Bluetooth interface may exchange transactional data with the vehicle 19, such as sending a signal that the box 21 and its contents have been received.

Further, in use, the box 21 may communicate the status of contents as well as the environment of the inner volume of the box 21 to the prescription processing system 30, for example, communicate messages regarding location, temperature, humidity, package status, delivery status, security panel access, removal or insertion of products, and the like. In other embodiments, this communication may send information from the box 21 to the vehicle 19.

Also in use, the box 21 may connect to a delivery vehicle 19, such as an autonomous vehicle by connecting to the autonomous vehicle systems or other delivery vehicle systems. For example, the box 21 may include an adapter that allow for environment control agents to be deployed into the inner volume of the box 21, connections to allow for transference of power from the vehicle 19 to the box 21 that may power the boxes 21. This may allow for seamless connection and disconnection from vehicle systems, wherein the connection between the vehicle system and the box 21 may be tracked; and the connection between the delivery vehicle system may be monitored for connective security.

In some embodiments, the box 21 may include a delivery encryption system comprising a blockchain for package tracking and authentication. The blockchain may include an ongoing chain hashed with key addresses along the chain of custody, including hashing with a seller private key address, a courier private key address and a buyer private key address, but not limited thereto. Here, a blockchain registers contents such as a medication or other pharmacy item and/or other grocery items to be delivered and placed within the inner volume of the box 21; and registers and authenticates the contents within the inner volume as the box 21 moves through a supply chain or otherwise between locations of interest.

In embodiments, the blockchain associated with the contents placed in the box 21 dynamically adjusts to account for the cold chain status of the product 50 as the smart package 10 moves through a supply chain. The chain block of a delivery encryption system may track and authenticate each of the contents, for example, both pharmacy and non-pharmacy items in the box 21.

In some embodiments, if the consumer 15 has availability for refills, the prescription processing system 30 can track the refill information and notify the consumer, based on assumed use according to the prescription, for example, 2 pills per day, when a refill is being processed. Automatic replenishment of a prescription is possible based on quantity, frequency, so on. When refills are performed in this manner, the refill medication may likewise be bundled with other items for delivery under a single order in a similar manner as described in other embodiments. In some embodiments, automatic replenishment is derived from replenishment data included in an original prescription blockchain.

Accordingly, at block 224, the prescription processing system 30 tracks refill dates and/or other refill information based on usage or consumption of prescribed medication. The prescription processing system 30 may generate an alert that is sent to the pharmacy computer 44 if determined usage amounts are different than a predetermined threshold or prescription amount.

At block 226, the pharmacy 14 may designate a refill in accordance with the prescription processing system. In some embodiments, the prescription processing system 30 tracks the pharmaceutical information, which includes a refill status that can be automatically tracked and fulfilled by the system 30.

At block 228, the doctor may receive at the doctor's computer 42 an alert that the pharmacy is initiating a refill in according with refill instructions generated by the prescription processing system 30. At block 230, the consumer 15 may approve the received delivery. The bundle may be created according to customer specific orders. The customer can turn away or reject a product or accept a product. The refill is tracked and staged but refills will be sent by customer request but not before refill date stated in prescription information.

In some embodiments, once a prescription has been submitted by the doctor or affiliation to the pharmacy 14, a blockchain of the patient's prescription may be created as described above. Here, any and all replenishments or refills for the prescription may also be included with this blockchain. From this refill information, a peer-to-peer ledger system and the application used by the retailer for this service will parse messages to the consumer patient 15 when a prescription requires a refill. This may be done at the suggested time of refill by the doctor and their affiliates, or may be accomplished by specific defined time windows by the retailer and a corresponding database or other repository accessible for performing the required functions.

When a prescription requires refill or replenishment and the customer has agreed to have this refilled or replenished, this may initiate a subsequent block, which will share information relating to the prescription with the pharmacy 14. The pharmacy will have the same functionality for procurement of the prescription, as well as a referral system.

In summary, automatic replenishment may be accomplished through data structured into a prescription blockchain. When the prescription has depleted or expired, a customer may be notified through the peer-peer ledger system of a prescription need. When selected by the patient, the prescription may be automatically replenished through the blockchain information, which will allow the prescription to be transmitted to a pharmacy. The pharmacy can then complete the processing of the prescription and send notification to the patient; or automatic delivery may also be featured within the blockchain data.

In some embodiments, information, for example, described in examples herein, may be shared, executed, and distributed through the application. In some embodiments, information, for example, described in examples herein, may be shared, executed, and distributed through the blockchain system by way of a peer-peer ledger system, identifiers, profiles, and keys.

In some embodiments, systems and methods provided that allows for the processing of a prescription through the blockchain's peer-peer ledger system where a patient has control of their records, allowing a prescriber of medication to transmit a prescription to the peer-peer ledger system. The patient can review the prescription from the peer-peer ledger system. The patient can select pharmacy providers from the peer-peer ledger system, which has a network of pharmacy providers with criteria, such as location, time, availability. Then, the patient can select from methods for pickup, delivery, or a trusted pickup. The methods may contain information on the entity stored on the peer-peer ledger system along with criteria, such as location, time, availability, and so on.

As described above, additional purchases may be added to a prescription as part of a delivery. Here, products and prescriptions may have blockchain identifiers that may be processed to determine similar products for recommendations. When selected by the customer, additional items may be added to the prescription delivery or pickup as a bulk order.

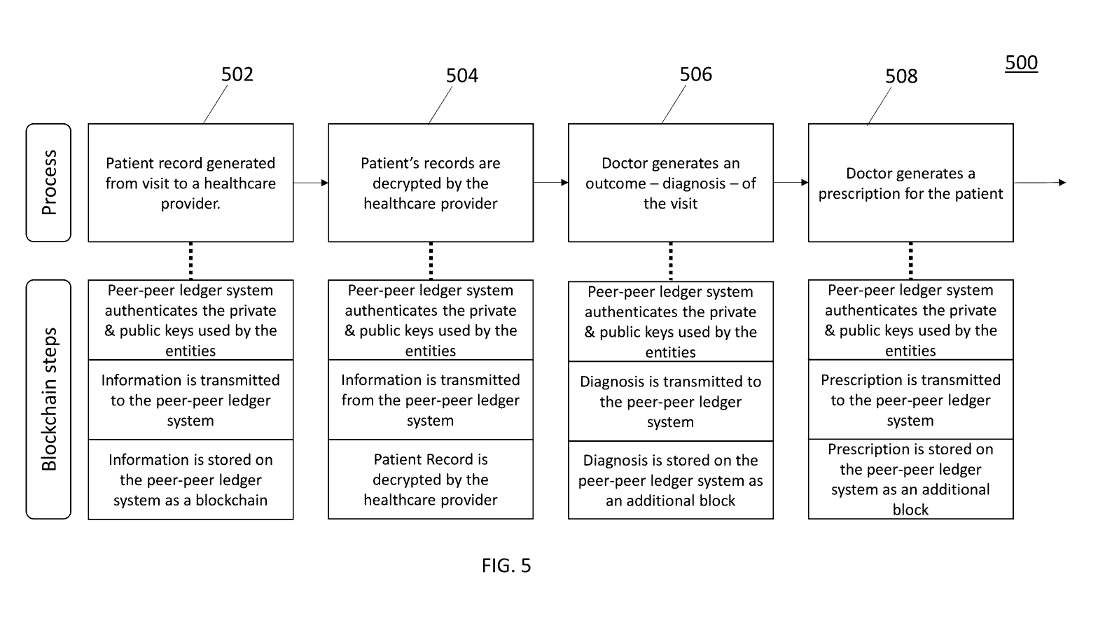

FIG. 5 is a process flow for processing patient records and prescription data, in accordance with some embodiments. As described in some examples, a process 500 may include method steps of generating (502) a patient record related to a visit to a healthcare provider, decrypting (504) the patient record, generating (506) by a doctor computer an outcome of the visit, and generating (508) a prescription for the patient. Some or all steps 502, 504, 506, 508 may include a set of blockchain steps performed by a blockchain system include special purpose processors and memories, illustrated in FIG. 5.

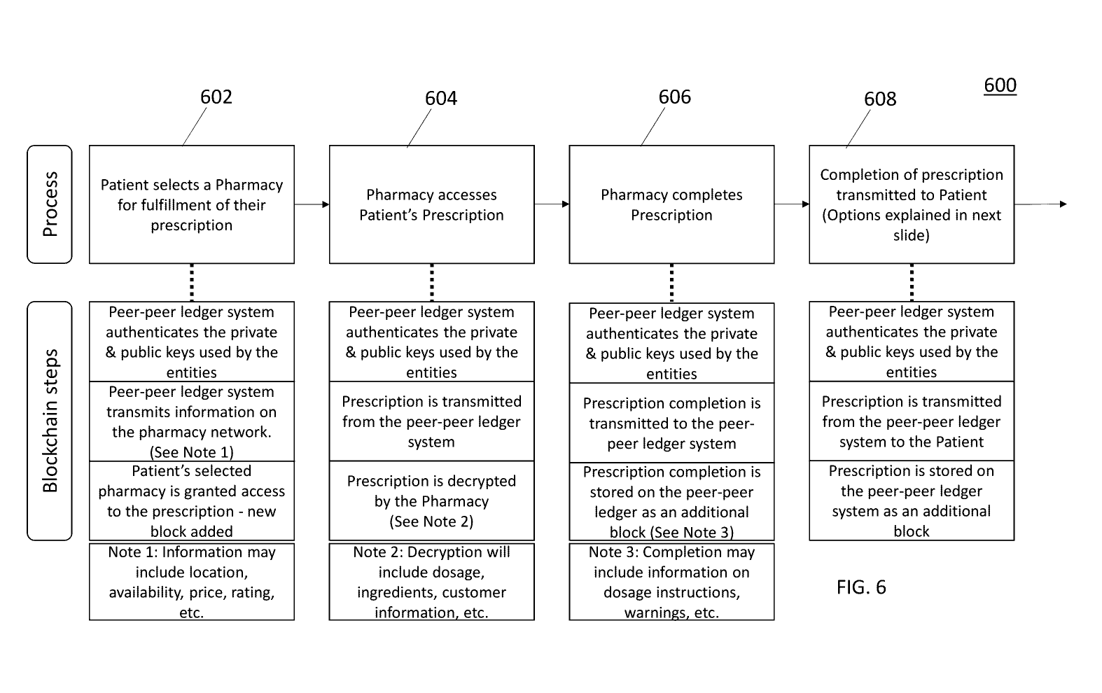

FIG. 6 is a process flow for pharmacy fulfillment of a prescription, in accordance with some embodiments. As described in some examples, a process 600 may include method steps of selecting (602) by a patient at a computer a pharmacy for fulfilling a prescription, accessing (604) by a selected pharmacy the patient prescription, completing (606) at the pharmacy computer the prescription, and completing (608) the prescription transmitted to the patient. Some or all steps 602, 604, 606, 608 may include a set of blockchain steps performed by a blockchain system include special purpose processors and memories, illustrated in FIG. 5.

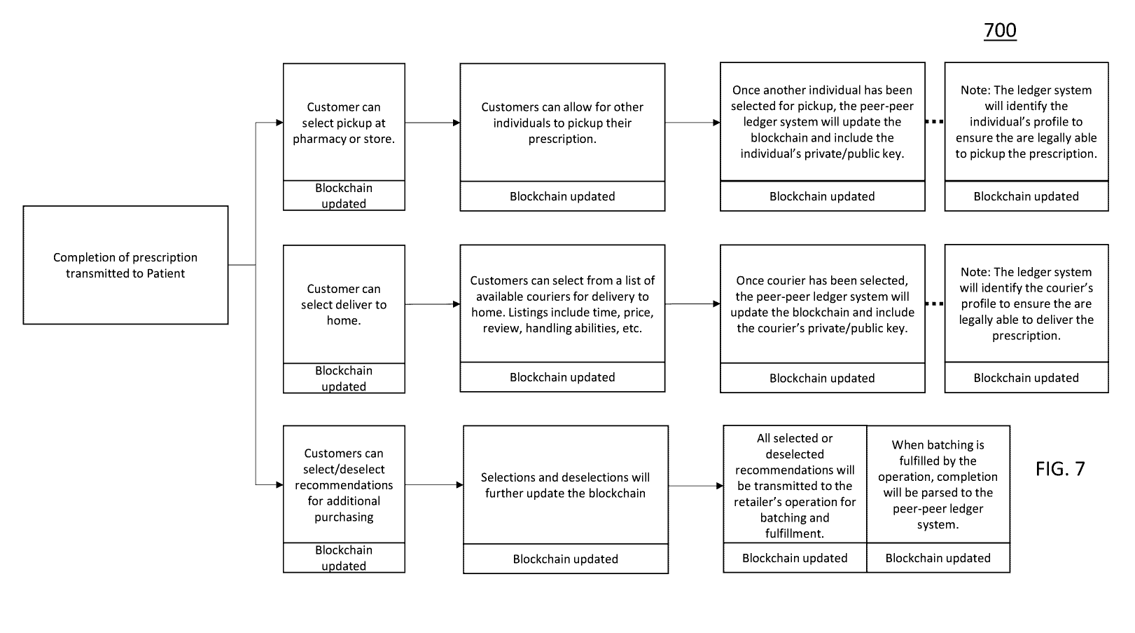

FIG. 7 is a patient decision process flow after a prescription fulfillment, in accordance with some embodiments. A process 700 may be performed after the prescription fulfillment process 600 illustrated and described with respect to FIG. 6, in particular, after completion of a prescription that is electronically transmitted from a prescription fulfillment computer for example at a pharmacy to a patient's computer, for example, laptop computer, smartphone, and so on. In each step of the process 700, a related blockchain is updated, for example, processed at a hardware computer.

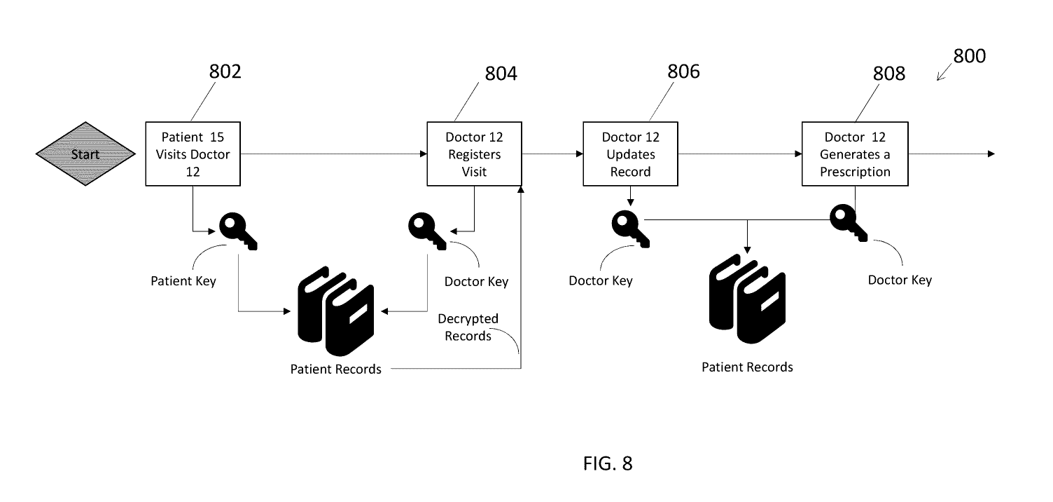

FIG. 8 is a process flow for a prescription fulfillment, in accordance with some embodiments. In describing FIG. 8, reference may be made to elements of FIGS. 1-7. As described in some examples, a process 800 may include method steps of a patient 15 visiting (802) a doctor 12, the doctor 12 registering (804) the visit, the doctor 12 updating (806) a patient record, and the doctor 12 generating (808) a prescription. Steps 802-808 may be similar to steps 502-508 of FIG. 5, and therefore, details are omitted due to brevity. A security key, e.g., a patient key or doctor key, is required at each step 802-808 to complete the respective step where accessing or decrypting/encrypting patient records is concerned. The process flow may include the use of a system comprising a blockchain for authentication. The blockchain may include an ongoing chain hashed with key addresses along the chain of custody, including hashing with a private key address, but not limited thereto. Here, a blockchain registers visit-related information, records, prescription details, and/or other information exchanged in the process 800.

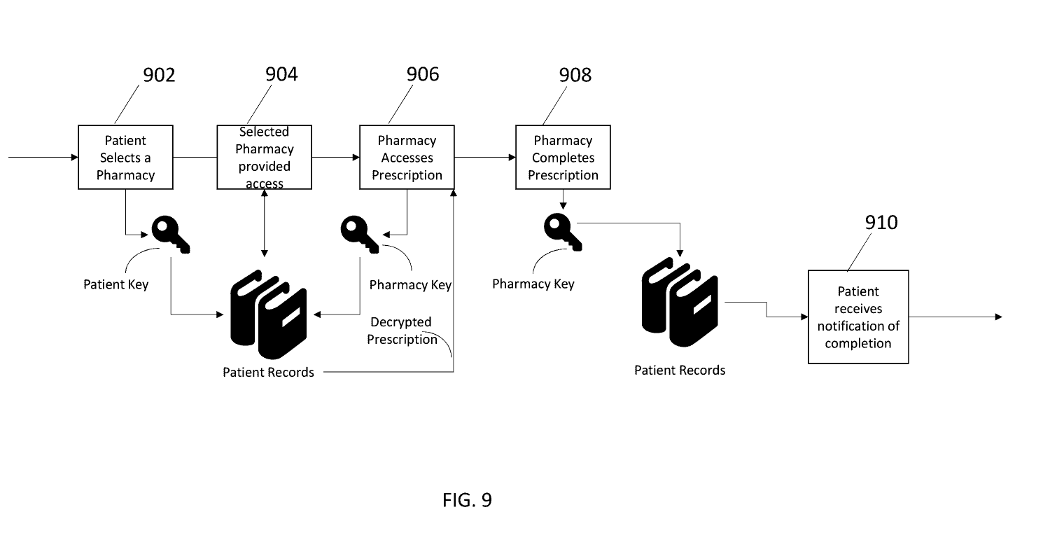

FIG. 9 is a process flow for a prescription fulfillment, in accordance with some embodiments. In describing the process 900 of FIG. 9, reference may be made to elements of FIGS. 1-8.

As described in some examples, a process 900 may include method steps of a patient 15 selecting (902) a pharmacy, which may occur after performing the process 800 of FIG. 8. The process 800 may further comprising providing access (904) by the selected pharmacy, accessing (906) a prescription, completing (908) the prescription, and receiving (910) a notification of the prescription. Steps 902-908 may be similar to steps 602-608 of FIG. 6, and therefore, details are omitted due to brevity. A security key, e.g., a patient key or doctor key, is required at each step 902-908 to complete the respective step where accessing or decrypting/encrypting patient records is concerned.

An example of an operation performed by the method steps of FIGS. 8 and 9 may include the following. Secure processing of sensitive patient data may be performed by an electronic device at a first location, for example, as the patient visiting the doctor's office. An electronic device at the doctor's office decrypts and accesses the patient's record. The doctor submits any new records to the patient's record through the peer-peer ledger system. The doctor submits a prescription to the peer-peer ledger system. The patient selects a pharmacy from the peer-peer ledger system and provides access to this pharmacy for prescription processing. The pharmacy completes and notifies the patient of the completed prescription through the peer-peer ledger system. The retailer suggests additional products for purchasing by identifying the prescription identifier and comparing its stored information with products. The patient selects methods for pickup or delivery through the peer-peer system, which may allow for specific access to the record. Further, the pharmacy and the retailer establishment are also communicated and transmitting with the same peer-peer ledger system. This may also include doctors and patients and/or other interested parties.

The systems and methods in accordance with some embodiments allows for the transmission, communication, procurement, and authentication of prescription information to be accomplished by a cryptography system facilitating a peer-peer ledger system, which controls access to the records and allows for iterations to be made. It is not limited to doctors and patients but will allow access for doctors, patients, pharmacies, couriers, and retailer.

The systems and methods in accordance with some embodiments generates alerts and messages from updates made to the peer-peer ledger system where the peer-peer ledger system communicates updates directly with authorized entities, which may include patients, pharmacies, doctors, retailer, couriers, and so on.

The systems and methods in accordance with some embodiments allows the peer-peer ledger system to store and distribute information on pharmacies available for processing the prescription, which may include information on location, pricing, availability, etc.

The systems and methods in accordance with some embodiments allow for blockchain product identifiers to contain data on the temperature requirements for a given product, It may further distribute this information to a temperature controlled device through active scanning, barcode scanning, private-key scanning, peer-peer ledger transmission, etc. Once the device has identified the product and its temperature requirements, it may control the temperature with its technology; all of which may be further distributed to the peer-peer ledger system to include real-time status, etc.

The systems and methods in accordance with some embodiments allow for pharmacy inventory levels (availability) to be transparent to the patient when selecting a pharmacy; further, the recommendation engine determines products for recommendation based on the prescriptions product identifier, which will include availability of items. All additions made to the order will further update the original prescription blockchain.

As will be appreciated by one skilled in the art, concepts may be embodied as a device, system, method, or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Computer program code for carrying out operations for the concepts may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Concepts are described herein with reference to flowchart illustrations and/or block diagrams of methods, apparatus (systems) and computer program consumer goods according to embodiments. It will be understood that each block of the flowchart illustrations and/or block diagrams, and combinations of blocks in the flowchart illustrations and/or block diagrams, can be implemented by computer program instructions. These computer program instructions may be provided to a processor of a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, cloud-based infrastructure architecture, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program consumer goods according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While concepts have been shown and described with reference to specific preferred embodiments, it should be understood by those skilled in the art that various changes in form and detail may be made therein without departing from the spirit and scope as defined by the following claims.

What is claimed is:

1. A method for home delivery of prescribed medication, comprising:
processing a prescription for a medication at a first location;
purchasing at least one item of interest at a second location; and
combining the prescribed medication and the at least one item of interest under a same delivery to a third location determined by a user of the prescribed medication and at least one item of interest.

2. The method of claim 1, wherein the first location is a pharmacy and the second location is a retail establishment.

3. The method of claim 2, wherein the pharmacy and the retail establishment are at a same location, or are the same.

4. The method of claim 1, further comprising:
sending the prescription via electronic communications from a doctor's office to the first location; and
generating a recommendation for other items of interest based on the prescription.

5. The method of claim 1, further comprising:
generating an automatic replenishment of the prescription based on the prescription.

6. The method of claim 1, further comprising:
generating an alert that is sent to the first location when an inventory supply falls below a predetermined threshold.

7. The method of claim 1, further comprising:
identifying a plurality of pharmacies available for processing the prescription; and
comparing a current location of the user and the first location to identify a pharmacy of the plurality of pharmacies for processing the prescription.

8. The method of claim 1, further comprising delivering the prescribed medication and the at least one item of interest in a temperature controlled device.

9. The method of claim 8, further comprising a delivery encryption system comprising a blockchain for package tracking and authentication.

10. The method of claim 1, further comprising delivering the prescribed medication and the at least one item of interest in an autonomous vehicle.

11. A prescription processing system, comprising:
an inventory determination processor that monitors an availability of a medication for prescribing to a recipient; and
a related products generator that determines items that may be combined with a prescription of the medication and other items that can be delivered together under a same shipping order to the recipient.

12. The prescription processing system of claim 11, further comprising a pharmacy determination processor that identifies a plurality of pharmacies may be available for prescribing a medication.

13. The prescription processing system of claim 11, further comprising a product recommendation processor that compiles data related to buying habits of the recipient and compares the data to the medication prescription to generate recommended buying options.

14. The prescription processing system of claim 11, further comprising a medication refill tracking processor that monitors refill dates regarding the prescription.

15. The prescription processing system of claim 11, wherein the system determines a delivery by combining the prescribed medication at a first location and the other items at a second location, and wherein the delivery is made to a third location.

16. The prescription processing system of claim 1, further including a blockchain processor for exchanging delivery information with one or more supply chain entities, wherein the blockchain processor is part of or in communication with a ledger system for communicating with one or more electronic devices of a customer, doctor, pharmacy, courier, and supply chain entities.

17. A method for home delivery of prescribed medication, comprising:
processing a prescription for a medication;
determining whether to purchase at least one item of interest;
combining the prescribed medication and the at least one item of interest under a same delivery to a predetermined location in response to determining that the at least one item of interest is purchased; and
delivering on the prescribed medication to the predetermined location in response to determining that the at least one item of interest is not purchased.

18. The method of claim 17, wherein the medication is at a first location and the at least one item of interest is at a second location.

19. The method of claim 17, further comprising:
generating an automatic replenishment of the prescription based on the prescription.

20. The method of claim 17, further comprising:
generating an alert when an inventory supply falls below a predetermined threshold.
